(19) United States
(12) Patent Application Publication
(10) Pub. No.: US 2025/0380931 A1
(43) Pub. Date: Dec. 18, 2025

(54) AN IMPROVED PROBE TOOL FOR ACQUISITION OF A BIOLOGICAL SAMPLE

(71) Applicant: LIFELINE ROBOTICS A/S, Odense S (DK)

(72) Inventor: Andreas Holmetoft LYDER, Odense S (DK)

(21) Appl. No.: 18/878,207

(22) PCT Filed: Jun. 29, 2023
(86) PCT No.: PCT/EP2023/067793

§ 371 Date: Dec. 23, 2024

(30) Foreign Application Priority Data
Jul. 1, 2022 (EP) 22182571.4

(51) Int. Cl. A61B 10/00

(52) U.S. Cl. CPC A61B 10/0051; A61B 10/0045

(57) ABSTRACT

The invention relates to a device (1) adapted to provide safe and consistent. sample obtaining from the nasal or oral cavity of a subject. The device comprises a holding element (HE) with an elongate base portion (HE2) adapted to be mounted in a housing (HO). the device further comprising at least one sensor (SENS), the sensor being adapted to measure a position of the elongate base portion when inserted into a canal (CA) of the housing. The housing further comprises a guiding mechanism (GM) positioned within the canal. adapted to guide and abut an outer surface (HESUR) of the elongate base portion of the holding element.

### FIELD OF THE INVENTION

The present invention relates to a device, system and method for the acquisition of a biological sample. In particular, the present invention relates to the safe acquisition of a sample from a subject, such as from the oral or nasal cavity of said subject.

### BACKGROUND OF THE INVENTION

Post-covid, automated systems for the safe obtainment of biological samples from subjects have emerged due to the scalability of performing sample obtaining with a reduced requirement for personnel, such as by using a robot with a sample obtaining device to obtain biological samples in vivo.

One example of such a system is disclosed in WO 2021/228808 “A sample obtaining device” disclosing a tool suitable for obtaining samples from subjects by mounting said tool on e.g. a robotic arm.

The tool as disclosed in WO 2021/228808 lacks essential features to be sufficiently safe for automated use and further improvements may be needed to overcome regulatory requirements. To elaborate, the tool as disclosed is based on a rigid system with static friction and with a rigid force sensor, wherein the force compliance is based on a force measurement, force measurement data being used to control the speed of the actuators on the robotic arm, via software, on which the tool is mounted. Should the software fail, the actuators would stop, rendering the system rigid in a stopped position. Further, the tool as disclosed were designed to envelope at least a portion of the swab, wherein at least some of the compliance of the swab would be reduced, as the swab moved into the tool.

Hence, an improved sample-obtaining device for automated sample obtaining would be advantageous, and in particular, a more efficient and/or reliable sample-obtaining device would be advantageous.

### OBJECT OF THE INVENTION

It is a further object of the present invention to provide an alternative to the prior art. In particular, the present inventions provides a safe and scalable device, method and system for the acquiring of biological samples from an oral or nasal cavity of a subject.

### SUMMARY OF THE INVENTION

Thus, the above-described object and several other objects are intended to be obtained in a first aspect of the invention by providing a device for acquisition of a biological sample from an oral or nasal cavity of a subject, the device comprising:
a holding element, the element comprising:
   a sample element holding portion, adapted to releasably fixate an end of an associated elongate sample obtaining element, such as a swab, opposite a sample obtaining end of the associated elongate sample obtaining element,
   an elongate base portion having a longitudinal axis coaxial to a length axis of said elongate base portion, the elongate base portion being connected to the sample element holding portion at a first end of the elongate base portion, the elongate base portion further having at least a first depression along an outer surface,
a housing, the housing comprising:
   a canal with an opening, wherein the opening is adapted to receive a second end of the elongate base portion and the canal is adapted to enclose at least a portion of the elongate base portion,
   a guiding mechanism adapted to abut and guide the outer surface of the elongate base portion of the holding element within the canal,
at least a first sensor, and when the elongate base portion is positioned within the canal, and
   the at least a first sensor is adapted to sense a position of the elongate base portion of the holding element relative to the canal, and wherein the guiding mechanism is adapted to provide a resistance force RF, between the canal and the elongate base portion of the holding element when the holding element is translated within the canal of the housing, wherein the RF is coaxial to the longitudinal axis of the elongate base portion and said RF varies depending on the position of the elongate base portion relative to the canal.

The invention is particularly, but not exclusively, advantageous for obtaining biological samples from a subject, such as a patient, in a safe manner. In a preferred embodiment, the device is adapted to be mounted on a peripheral positioning device, such as a robotic arm or actuated lever, and wherein the device ensures that the subject is not harmed by excess force during the obtaining of the sample. As an example, the device is suitable for obtaining mouth swabs or nasal swabs from a patient, such as for sampling the patient for bacterial or viral infections.

In the context of the present invention, an associated elongate sample-obtaining element is to be understood as a mouth swab, nasal swab or other suitable and disposable means for obtaining samples from within a cavity of a subject. It is further to be understood, that an associated sample-obtaining element does not need to be elongate, but that this is a standard form of such sample devices. In some embodiments, the device is adapted to receive and releasably fixate other types of associated disposable sample obtaining elements.

Thus, the invention is particularly advantageous for near-patient automated care and sample obtaining, i.e. wherein at least a part of the device is within few centimetres or millimetres of a patient, while ensuring that the device prevents any harm from being performed to said patient, by e.g. excess movement from said peripheral positioning device. It is to be understood, that the associated elongate sample obtaining element may have a flexible shaft portion enabling movement of the sample obtaining end, whereas substantial force in an axial direction is translated to the holding element, i.e. if the associated sample obtaining element is pushed into the tissue of a patient, the elongate base portion may translate at least some of the resulting force into a backwards translational movement of the holding element, relative to the housing, i.e. the elongate base portion moves further into the canal, thus reducing force applied to the tissue to an acceptable force.

In a preferred embodiment of the invention, the device is configured to provide 0.1 N to 2.0 N of force to the associated sample-obtaining element, more preferably 0.4 to 1.5 N of force and most preferably 0.5 to 1.0 N of force.

In a more preferred embodiment of the invention, the device consistently obtains samples at 0.6 to 0.8 N of force.

It is to be understood that the elongate base portion has at least a first surface along a length of a periphery of said elongate base portion. As an example, if the cross section of the elongate base portion is round or elliptical, there is one side, if the cross section of the elongate base portion is triangular, there is three sides, if the cross section of the elongate base portion is rectangular or square, there is four sides etc. It is further to be understood, that the elongate base portion is adapted to be received within the canal, i.e. a cross-sectional shape of the elongate base portion has a complimentary shape to that of a cross section shape of the canal.

In a preferred embodiment, a cross section of the canal of the housing and the cross section of the elongate base portion are substantially similar, i.e. the one or more surfaces of the elongate base portion are substantially parallel to one or more inner surfaces of the canal.

In some embodiments, the elongate base portion may be enabled to rotate around a longitudinal axis, within the canal.

In a preferred embodiment, the elongate base portion has a cross sectional geometry preventing said elongate base portion to rotate within the canal, around a longitudinal axis of the elongate base portion.

In another preferred embodiment of the invention, the elongate base portion comprises a semi-rigid or rigid material.

In yet another preferred embodiment, the elongate base portion forms a semi-rigid or rigid, molded unitary member.

In an advantageous embodiment of the invention, the holding element comprises an anti-bacterial and/or anti-viral surface.

In some embodiment, the holding element is reusable, i.e. suitable for obtaining a plurality of samples from two or more subjects, and wherein a surface of at least the sample element holding portion are substantially flush, i.e. wherein at least the sample element holding portion are suitable for disinfecting and substantially without crevices, recesses or other depressions.

In some embodiments, the opening of the canal may be wider or narrower than the canal.

In some embodiments, the opening of the canal may be a removable opening, such as a threaded cap or a snap-fit cap.

In the context of the present invention, the depression of the outer surface of the elongate base portion may be a recess, such as an elongate recess. In some embodiments, the elongate base portion has a substantially round or elliptical cross section, i.e. a substantially round or elliptical outer surface, and wherein the depression may cover some or all of a periphery said outer surface along a length of the elongate base portion.

In other embodiments, the depression is positioned on at least a first surface of a plurality of surfaces of the elongate base portion.

It is further to be understood, that the depression is to be understood as a portion of the elongate base portion, wherein a cross sectional diameter of the elongate base portion in a plane of the depression, is smaller than a cross sectional diameter of the elongate base portion at a plane adjacent to said depression.

In some embodiments, the canal may have a slightly larger diameter than the elongate base portion and wherein one or more surfaces of the canal are configured to avoid abutting the one or more surfaces of the elongate base portion.

In a preferred embodiment, the opening of the canal is adapted to stop the holding element from translating fully into the canal, as a cross sectional diameter of the opening is less than a cross sectional diameter of the sample element holding portion, i.e. as the elongate base portion is moved fully into the canal, at least a portion of the sample element holding portion abuts an outer surface of the opening.

In other embodiments, a cross sectional geometry of the opening is different from a cross sectional geometry of the sample element holding portion, the different geometries of the two acting as an end-stop, wherein the sample element holding portion are configured to as to not fit within said opening.

In the context of the present invention, the guiding mechanism is to be understood as one or more means for receiving and abutting an at least first outer surface of the elongate base portion. The guiding mechanism may be a friction material positioned within the canal, wherein a frictional force between an outer surface of the elongate base portion and the friction material is less or more than a friction between an inner surface of the canal and the outer surface of the elongate base portion.

In other embodiments, the guiding mechanism may comprise a biased surface, such as a leaf spring mechanism, and wherein a protruding surface of the leaf spring abuts an outer surface of the elongate base portion. This embodiment of the invention is particularly advantageous for providing a variable resistance force between the guiding mechanism and the elongate base portion, as a spring force between said leaf spring and the surface decreases when a plane of the leaf spring and a plane of the depression coincides.

In some embodiments, the elongate base portion and the guiding mechanism are formed so as to provide a friction fit, said friction fit having a resistance force FFRF, and wherein said FFRF is less than a force required to penetrate the tissue within a nasal cavity or oral cavity of a subject with an associated elongate sample obtaining element with a diameter of e.g. 0.5 cm or 0.2 cm.

Thus, it is to be understood that the depression on the outer surface of the elongate base portion is adapted to reduce the resistance force between the canal and/or guiding mechanism, as the elongate base portion is positioned in a sample position, i.e. a position wherein the device is ready to obtain a sample from a subject.

In the context of the present invention, the at least one sensor may be selected from one or more of a pressure sensor, pressure transducer, strain gauge, a position sensor, a force sensor, a piezo sensor, a fluid sensor or other suitable sensor. The at least one sensor is adapted to send a measuring signal.

It an advantageous embodiment of the invention, the sensor is adapted to measure force applied to the associated elongate sample obtaining element, wherein said force applied, during sample obtaining, translated through the associated elongate sample obtaining element, to the holding element, from the holding element to the canal and/or guiding mechanism; and wherein the sensor measures force applied to the guiding mechanisms from the holding element,; or force applied to a surface of the canal from the holding element; or a relative movement between the holding element and the canal of the housing.

In a preferred embodiment, the sensor is adapted to measure relative movement between the elongate base portion of the holding element and the canal of the housing as a result of force applied to the holding element through the associated elongate sample-obtaining element. In some embodiments, the sensor may further be adapted to convert translational movement of the elongate base portion into force, as Newton's second law states that the net force acting upon an object is equal to the rate at which its momentum changes with time. If the mass of the object is constant, this law implies that the acceleration of an object is directly proportional to the net force acting on the object, is in the direction of the net force, and is inversely proportional to the mass of the object. As an example, this can be done by plotting the frictional force between the elongate base portion and the canal and/or guiding mechanism, the weight of the moving parts of the device, i.e. the holding element and associated elongate sample-obtaining element and measure the acceleration of the elongate base portion relative to the canal, during sample obtaining.

In some embodiments, the sensor may be calibrated when/if the holding element is to be replaced.

It is further to be understood, that the sensor may be adapted to send a measured signal, either to processing means, such as computer with a processor, or to a controller device of a peripheral positioning device. It is further to be understood, that a stop signal may be transmitted either from the sensor or a computer connected to the sensor, if a force threshold is exceeded or if a positional threshold of the elongate base portion within the canal is exceeded.

In some embodiments, the device further comprises controlling means, adapted to receive a measurement signal from the at least one sensor and, based on measurements from the sensor, send a go and/or stop signal to a peripheral device. In some embodiments, the device is adapted with a threshold, such as a force, position or acceleration threshold, and wherein a measured force, position or acceleration exceeding said threshold triggers a stop signal to said peripheral device.

It is to be understood, that the controlling means may be a circuit board or computer, such as with a processer, and wherein program code on said controlling means enables the controlling means to execute at least a stop signal and/or a go signal.

In a preferred embodiment of the invention, the sensor and or controlling means are adapted to output a measured signal as measurement data such as force, position, acceleration data or velocity data based on a measured signal, to a peripheral device, enabling said peripheral device to evaluate said data during sample obtaining.

In the context of the present invention, resistance force RF is to be understood as any force, such as frictional force, which are to be exceeded for the elongate base portion to move, relative to the canal and/or guiding mechanism. In some embodiments, the resistance force is equal to a frictional force between the surface of the elongate base portion and the guiding mechanism. In other embodiments, the resistance force may be a product of frictional force and other force, such as a magnetic force, rolling friction or other resistance to movement between the guiding mechanism and the elongate base portion. Further, the resistance force is to be understood as a coaxial force, and wherein the resistance force is substantially parallel to a longitudinal axis of the device.

In a preferred embodiment, the resistance force varies along the length of the elongate base portion, at least due to the depression on the surface of said elongate base portion. Further, the depression ensures the position of the elongate base portion is constant, as a plane of the guiding mechanism are collateral to a plane of said depression, when no external force is applied to the holding element, i.e. when the guiding mechanism abuts the depression, the holding element is still until force is applied.

In another preferred embodiment of the invention, the device further comprises an interlocking element, the interlocking element adapted to be fixated to a peripheral positioning device, such as a robotic arm, the interlocking element further comprising mounting means adapted to mount the housing on the interlocking element, and wherein the at least one sensor is positioned within the interlocking element.

It is to be understood that the device is configured along a longitudinal axis wherein at least the associated elongate sample obtaining element, the holding element and the housing are coaxially aligned on said longitudinal axis. Thus at one end of the device, the sample obtaining end of the associated elongate sample obtaining element is positioned and at the opposite end of the longitudinal axis the housing or the interlocking element is positioned. In between the associated elongate sample obtaining element and the housing, the holding element is positioned. In some embodiments, the interlocking element is further aligned on said longitudinal axis of the device and wherein the housing is positioned between said interlocking element and the holding element.

It is further to be understood, that the interlocking element may be incorporated into the housing or be a singular unit between the housing and a peripheral position device.

Even further, it is to be understood, that the peripheral device may be one or more of a robotic arm or other actuated device adapted to move without any user actuation. In some embodiments, the peripheral movement device may be a user, such as a health care person, and wherein the interlocking element is adapted for suitable gripping from the hand of said user, and wherein the device is adapted to ensure correct pressure applied to the tissue of a subject from the user during sample obtaining.

In an advantageous embodiment of the invention, the device further comprises an electric motor adapted to rotate the housing relative to the interlocking element or the holding element relative to the housing or the associated elongate sample obtaining relative to the holding element so as to rotate the associated elongate sample obtaining element around a longitudinal axis.

This embodiment is particularly advantageous for ensuring, that the associated elongate sample obtaining element obtains a sufficient sample from the tissue of a subject, i.e. wherein the sample obtaining end of the associated sample obtaining element is rotated against the tissue of the subject.

In some embodiments, the electric motor is positioned within the housing, enabling the holding element to rotate relative to said housing. It is to be understood, that the electric motor may be adapted to rotate the canal of the housing or to rotate the guiding mechanism relative to the canal. Further, in some embodiment, the electric motor may be positioned in the guiding mechanism, adapted to rotate the holding element relative to said guiding mechanism.

In some embodiments, the electric motor is positioned within the interlocking element, enabling the housing to rotate relative to said interlocking element.

In some embodiments, the electric motor is positioned within the holding element such as between the elongate base portion and the sample element holding portion and wherein the electric motor is adapted to rotate the sample element holding portion relative to the elongate base portion.

It is to be understood, that in any of the above mentioned embodiments relating to the position of the electric motor, the rotating of the electric motor is translated into a rotation of the associated elongate sample obtaining element to ensure sufficient sampling of tissue, bacteria, virus or cells from a subject.

In a preferred embodiment of the invention, the electric motor is adapted to rotate when a sensor, such as said at least one sensor or a second sensor, measures translation of the elongate base portion within the canal from a first position to a second position, as a result of force applied to the holding element from the associated elongate sample obtaining element, preferably wherein the electric motor is engaged when the sample obtaining end of the associated sample obtaining element abuts tissue of the subject.

This embodiment is particularly advantageous for ensuring a high quality of the sample from the subject without stressing said subject. As one may imagine, having a rotating object positioned within a cavity may be stressful to a subject, whereas having a relatively still rotating object inserted would be less stressful and only engaging the rotation of the object after the object is without the field of vision of the subject reduces said stress.

It is to be understood that the at least one sensor may be adapted to measure translation of the elongate base portion. In other embodiments, the at least one sensor or a second sensor may be adapted to measure force applied to the elongate base portion. It is further to be understood, that a force applied to the elongate base portion is a force translated from the associated elongate sample obtaining element as the sample obtaining end comes into contact with tissue of the subject.

In some embodiments, the at least one sensor or a second sensor is adapted to measure force between the canal of the housing and the elongate base portion of the holding element. In other embodiments, the at least one sensor or a second sensor is adapted to measure force between the guiding mechanism of the housing and the elongate base portion of the holding element. In some embodiments, the device is adapted to measure both a translational movement of the elongate base portion relative to the canal and/or guiding mechanism and a second force, such as a frictional force between the elongate base portion and the canal and/or guiding mechanism.

In another preferred embodiment of the invention, the at least one sensor is an electromagnetic sensor, such as a hall sensor and the holding element further comprises a magnetic portion, the electromagnetic sensor being adapted to sense the position of the holding element within the canal of the housing.

In some embodiments, the magnetic element may be positioned within the elongate base portion of the holding element.

In some embodiments, the sensor may be adapted to reset a position of the holding element when the depression of the elongate base portion is collateral to the guiding mechanism, i.e. wherein a position of the holding element is zero, when said depression is positioned collateral to the guiding mechanism. It is further to be understood, that the at least one sensor may be calibrated to recognize the abovementioned position as a point of reference or benchmark position.

In some embodiments, the device is adapted to initiate sample obtaining as the elongate base portion of the inserted into the canal. In particular, the device may be adapted to initiate sample obtaining when the depression is positioned collateral to the guiding mechanism based on detection from the at least one sensor or a second sensor.

In other embodiments, the sample element holding portion may be adapted with a sensor, and wherein said sensor is adapted to detect the insertion of an associated elongate sample obtaining element in the sample element holding portion of the holding element. Further, the device may be adapted to initiate sample obtaining, such as by sending a signal, when the at least one sensor or a second sensor detects the insertion of an associated elongate sample obtaining element in the sample element holding portion.

In yet other embodiments, the at least one sensor is an optical sensor, such as an optical sensor configured to detect the reflection of a modulated light beam and calculating distance as a function of Time-of-Flight principle, the optical sensor being adapted to sense the position of the holding element within the canal of the housing. The time-of-flight principle is based on measuring the time it takes for a wave to travel from the optical sensor to an object, i.e. the holding element, and back to the optical sensor. Based on that data and wave propagation, the distance between the holding element and the optical element can be established. To increase accuracy and stability of distance measurements, the holding element may further comprise a surface normal to the modulated light beam.

In an advantageous embodiment of the invention, the elongate base portion of the holding element is tapered and wherein a cross-section of the elongate base portion in at least a section, increases from the second end towards the sample element holding portion.

It is to be understood, that the cross-section of the elongate base portion increases progressively, excluding the cross-section in a plane wherein the depression of the surface is located.

Thus, in some embodiments, the cross-section of the elongate base portion increases along a length of the elongate base portion until the depression, and wherein the cross-section decreases along a length of the depression. In some embodiments, the depression is a curved depression with a slope and wherein a cross-section of the elongate base portion is smallest at the centre of the depression.

In some embodiments, the resistance force between the elongate base portion and the guiding mechanism increases along a length of the elongate base portion, and wherein the resistance force decreases as the guiding mechanism abuts the depression of the elongate base portion.

In another advantageous embodiment of the invention, the cross-section of the elongate base portion of the holding element is noncircular, preferably the cross-section is substantially triangular and wherein the elongate base portion has three sides. This embodiment of the invention is particularly advantageous to ensure, that the elongate base portion is prohibited from rotating within the canal.

In a preferred embodiment, the cross-section of the elongate base portion is substantially triangular and wherein the elongate base portion has three sides. It is to be understood, that the guiding mechanism is adapted to abut each of said sides, so as to align said sides to said guiding mechanism.

In the abovementioned embodiments, it is further to be understood, that the depression may located on a single side or surface of the elongate base portion or on some or all sides of said elongate base portion. Further, in some embodiments, the depression may be along the periphery of the surface of elongate base portion including all sides of the elongate base portion, such as wherein the elongate base portion is substantially round or elliptic and wherein the depression has a narrower diameter than a diameter of the surface of said round or elliptic elongate base portion.

In a preferred embodiment of the invention, the guiding mechanism comprises at least a first and second set of rollers adapted to roll along the surface of the elongate base portion of the holding element. This embodiment is particularly advantageous to provide a device, wherein the elongate base portion is easy to insert within the canal of the housing. Further, the rollers may be adapted with a sensor, such as a sensor adapted to measure or detect any rotation of said rollers.

In another preferred embodiment, the guiding mechanism comprises three sets of rollers adapted to abutting three sides of the elongate base portion. This embodiment is particularly advantageous to align a peripheral orientation of the elongate base portion relative to a periphery of the canal of the housing.

In an advantageous embodiment of the invention, the guiding mechanism comprises at least a first set of rollers and wherein the at least first set of rollers are spring biased towards the surface of the elongate base portion of the holding element, with a spring force SF substantially lateral to the longitudinal axis of the elongate base portion, wherein the SF translates to the RF, an application force AF is coaxial to the longitudinal axis of the elongate base portion in a direction opposite to the RF, and to insert the elongate base portion into the canal of the housing, the AF exceeds the RF.

In another advantageous embodiment of the invention, the guiding mechanism comprises three sets of rollers adapted to abutting three sides of an outer surface of the elongate base portion of the holding element.

This embodiment is particularly advantageous for providing a device, wherein the resistance force is a result of an angle of the surface of the elongate base portion, the spring force and the friction of the rollers, i.e. wherein the resistance force increases as a cross sectional diameter of the elongate base portion increases and the springs are compressed. As an example, as the rollers roll along the surface and wherein the cross sectional diameter is substantially uniform, the resistance force is primarily a result of a frictional force of the rollers. When the cross sectional diameter decreases, the resistance force may be inverse, i.e. wherein the direction of the force is opposite. Thus, this embodiment provides a device wherein the resistance force of the holding element relative to the housing is in general a result of the geometric shape of an outer surface of the elongate base portion.

It is to be understood, that the springs may be positioned in between the rollers and an inner portion of the housing, i.e. wherein the springs are adapted to provide a spring force as the rollers are moved from within the canal towards an outer periphery of the housing.

In an advantageous embodiment of the invention, a plurality of spring biased sets of rollers are positioned around a periphery of the canal of the housing, preferably wherein the plurality of sets of rollers are positioned uniformly, at a distance from each other, in an array around an inner periphery of the canal.

In another advantageous embodiment of the invention, a friction force FF between the at least first and second set of rollers and the surface of the elongate base portion equals the RF, and wherein the FF increases as the holding element is introduced into the canal of the housing, said FF reducing when the depression in the surface of the elongate base portion and the rollers are collateral, as the holding element is introduced further into the canal of the housing. This embodiment is particularly advantageous for providing a device, wherein the resistance force is a result of a controlled friction within the at least first and second set of rollers. In some embodiments, the guiding mechanism comprises a single set of rollers and wherein the resistance force is a result of a friction of said set of rollers and a second frictional force between an outer surface of the elongate base portion and the guiding mechanism or an inners surface of the canal of the housing.

In yet another advantageous embodiment of the invention, the frictional force FF is electronically adjustable by providing braking means for at least one of the at least first and second set of rollers. This embodiment is particularly advantageous for providing a device, wherein the resistance force RF is equal to at least the FF and wherein the RF can be adjusted be regulating the frictional force of at least one of the sets of rollers. It is to be understood, that the guiding mechanism may comprise a controller and wherein said controller is adapted to adjust the FF by activation or release of the braking means, thus adjusting the RF during the translation of the elongate base portion within the canal of the housing. In some embodiments, the braking means may provide a device, wherein the surface of the elongate base portion is substantially uniform and wherein the depression can be excluded from said elongate base portion, as a variable resistance force can be achieved through activation and release of said braking means.

In a preferred embodiment of the invention, the device further comprises an output device connected to the at least one sensor, the output device adapted to send a signal to a peripheral device based on a measurement from said at least one sensor. It is to be understood, that the output device is in communication with the at least one sensor, such as a wired or wireless data communication between said output device and the at least one sensor. It is further to be understood, that the output device may be adapted to be in data communication with a peripheral device, such as a peripheral movement device or a computer, and wherein said output device may be configured to relay measurements from the at least one sensor to the peripheral device or e.g. to the braking means according to some embodiments of the invention. This embodiment of the invention is particularly advantageous for providing a device, wherein said device can provide continuous measurements from the at least one sensor, such as positional measurements of force measurements to e.g. a peripheral device. It is to be understood that such measurement may be used to control or monitor the sample obtaining process and/or to prevent the associated elongate sample obtaining element from causing harm to a subject during sample obtaining.

In another preferred embodiment of the invention, the device further comprises a second sensor, preferably wherein the guiding mechanism comprises a second sensor, the second sensor adapted to sense the elongate base portion moving relative to the canal of the housing. This embodiment is particularly advantageous for providing an ancillary safety mechanism or an additional means of measuring movement or force between the holding element and the housing resulting from the application of a force to the associated elongate sample obtaining element. In some embodiments, the second sensor may measure a force or acceleration, such as a frictional force or acceleration between the guiding mechanism and the elongate base portion. In some embodiments, the second sensor may be in data communication with a controller. In other embodiments, the second sensor may circumvent a controller of the device and provide a signal or measurement data directly to a peripheral device.

In yet another preferred embodiment of the invention, the holding element and the elongate sample obtaining element are one unitary element. This embodiment is particularly advantageous for providing an even more hygienic device, i.e. wherein crevices and recesses may be reduced and/or wherein the unitary element may be disposable. It is to be understood, that the unitary element may be a pre-packaged sterile element, and wherein the unitary element is disposed after sample obtaining.

In a second aspect, the invention relates to a device for acquisition of a biological sample from an oral or nasal cavity of a subject, the device comprising:
a holding element, the element comprising:
a sample element holding portion, adapted to releasably fixate an end of an associated elongate sample obtaining element, such as a swab, opposite a sample obtaining end of the associated elongate sample obtaining element,
an elongate base portion having a longitudinal axis coaxial to a length axis of said elongate base portion, the elongate base portion being connected to the sample element holding portion at a first end of the elongate base portion,
a housing, the housing comprising:
a canal with an opening, wherein the opening is adapted to receive a second end of the elongate base portion and the canal is adapted to enclose at least a portion of the elongate base portion,
a guiding mechanism adapted to abut and guide the outer surface of the elongate base portion of the holding element within the canal,
at least one sensor, and when the elongate base portion is positioned within the canal, and
   the at least one sensor is adapted to measure a position of the elongate base portion of the holding element relative to the canal, and wherein the guiding mechanism is adapted to provide a resistance force RF, between the canal and the elongate base portion of the holding element when the holding element is translated within the canal of the housing, wherein the RF is coaxial to the longitudinal axis of the elongate base portion and said RF varies depending on the position of the elongate base portion relative to the canal.

This embodiment is particularly advantageous for providing a device with a variable resistance force RF, wherein said variable resistance is a result of e.g. friction between the canal or guiding mechanism and a surface of the elongate base portion of the holding element.

In a third aspect, the invention relates to a kit of parts comprising the device according to any of the first and second aspects of the invention, wherein an elongate sample obtaining element are prefixated to the holding element. This aspect is particularly advantageous for providing a sterile kit, wherein at least a first fixated elongate sample obtaining element is ready for use without the need for a user to insert the elongate sample obtaining element into the holding portion and or wherein the elongate base portion of the holding element is positioned within the housing of the canal.

In a preferred embodiment of the invention, the kit of parts comprises a housing according to the first or second aspect of the invention, the kit further comprising one or more disposable holding elements and sample obtaining elements, and wherein the one or more disposable sample obtaining elements are prefixated to respective disposable holding elements. This embodiment is particularly advantageous for providing a kit, wherein at least each set of holding elements and sample obtaining elements are sterile prior to use, i.e. sample obtaining.

In another preferred embodiment of the invention, the kit of parts comprises a housing and interlocking element according to the first or second aspect of the invention, the kit further comprising one or more disposable holding elements and sample obtaining elements, and wherein the one or more disposable sample obtaining elements are prefixated to respective disposable holding elements. This embodiment is particularly advantageous for providing a kit, wherein at least each set of holding elements and sample obtaining elements are sterile prior to use.

In yet another preferred embodiment of the invention, the kit of parts comprises a housing with an integrated interlocking element, the kit further comprising one or more disposable holding elements and sample obtaining elements, and wherein the one or more disposable sample obtaining elements are prefixated to respective disposable holding elements. This embodiment is particularly advantageous for providing a kit, wherein at least each set of holding elements and sample obtaining elements are sterile prior to use.

In a fourth aspect, the invention relates to a method of acquiring a biological sample from an oral or nasal cavity of a subject, the method comprising:
providing an interlocking element, the interlocking element being positioned on a peripheral positioning device,
providing a housing mounted on the interlocking element, the housing comprising:
a canal with an opening, and
a guiding mechanism,
providing a holding element, the holding element comprising
a sample element holding portion adapted to hold an associated elongate sample obtaining element, and
an elongate base portion connected to the sample element holding portion, the elongate base portion being tapered, increasing in cross-section from an end opposite from the sample element holding portion towards the sample element holding portion and a depression at a position between the second end and the sample element holding portion,
providing at least one sensor, said sensor being adapted to detect a position of the elongate base portion relative to the canal, and further adapted to communicate with a peripheral device, preferably wherein the sensor is in communication with the peripheral positioning device,
fixating the associated elongate sample obtaining element to the sample element holding portion,
introducing the elongate base portion into the opening of the canal until the depression of the elongate base portion is collateral with the guiding mechanism,
moving the associated elongate sample obtaining element towards and into an oral or nasal cavity of a subject by moving the peripheral positioning device from a first position to at least a second position, and
obtaining a sample from the oral or nasal cavity of the subject and moving the peripheral positioning device from the at least second position to a position different from the second position, and
stopping the peripheral positioning device from moving based on a signal from the at least one sensor.

In an alternative embodiment of the invention, the method comprises:
providing a device according to the first or second aspect of the invention,
fixating the associated elongate sample obtaining element to the sample element holding portion,
introducing the elongate base portion into the opening of the canal until the elongate base portion is collateral with the guiding mechanism,
moving the associated elongate sample obtaining element towards and into an oral or nasal cavity of a subject by moving the peripheral positioning device from a first position to at least a second position, and
obtaining a sample from the oral or nasal cavity of the subject and moving the peripheral positioning device from the at least second position to a position different from the second position, and
stopping the peripheral positioning device from moving based on a signal from the at least one sensor.

In a preferred embodiment of the invention, the method further comprises:
providing an electric motor adapted to rotate the housing relative to the interlocking element or the holding element relative to the housing or the associated elongate sample obtaining element relative to the holding element, and
activating the electric motor when the peripheral positioning device is at least in the second position.

In a fifth aspect, the invention relates to a system for acquisition of a biological sample from an oral or nasal cavity of a subject, the system comprising:
a holding element, the element comprising:
a sample element holding portion, adapted to releasably fixate an end of an associated elongate sample obtaining element, such as a cotton swab, opposite a sample obtaining end of the associated elongate sample obtaining element,
an elongate base portion connected to the sample element holding portion at a first end of the elongate base portion, the elongate base portion further having at least a first depression along an outer surface,
a housing, the housing comprising:
a canal with an opening, wherein the opening is adapted to receive a second end of the elongate base portion and the canal is adapted to enclose at least a portion of the elongate base portion,
a guiding mechanism adapted to abut and guide the outer surface of the elongate base portion of the holding element within the canal,
an interlocking element, the interlocking element adapted to be fixated to a peripheral positioning device, the interlocking element further comprising:
mounting means adapted to mount the housing on the interlocking element,
at least one sensor positioned within the interlocking element, the sensor adapted to communicate with a peripheral device, preferably the peripheral positioning device,
   wherein the at least one sensor is further adapted to sense a coaxial position of the elongate base portion of the holding element relative to the canal, the guiding mechanism is adapted to provide variable resistance between the housing and the surface of the elongate base portion of the holding element, when the holding element is translated within the canal of the housing as the associated elongate sample element obtains a sample from a subject and the system is adapted to stop the peripheral positioning device based on a signal from the at least one sensor.

In an alternative embodiment of the invention, the system comprises:
a holding element, the element comprising:
a sample element holding portion, adapted to releasably fixate an end of an associated elongate sample obtaining element, such as a cotton swab, opposite a sample obtaining end of the associated elongate sample obtaining element,
an elongate base portion connected to the sample element holding portion at a first end of the elongate base portion, the elongate base portion further having at least a first depression along an outer surface,
a housing, the housing comprising:
a canal with an opening, wherein the opening is adapted to receive a second end of the elongate base portion and the canal is adapted to enclose at least a portion of the elongate base portion,
a guiding mechanism adapted to abut and guide the outer surface of the elongate base portion of the holding element within the canal,
an interlocking element, the interlocking element adapted to be fixated to a peripheral positioning device, the interlocking element further comprising:
mounting means adapted to mount the housing on the interlocking element,
at least one sensor positioned within the interlocking element, the sensor adapted to communicate with a peripheral device, preferably the peripheral positioning device, and
   wherein the at least one sensor is adapted to sense a position of the elongate base portion of the holding element relative to the canal, and wherein the guiding mechanism is adapted to provide a resistance force RF, between the canal and the elongate base portion of the holding element when the holding element is translated within the canal of the housing, wherein the RF is coaxial to the longitudinal axis of the elongate base portion and said RF varies depending on the position of the elongate base portion relative to the canal as the associated elongate sample element obtains a sample from a subject and the system is adapted to stop the peripheral positioning device based on a signal from the at least one sensor.

In another alternative embodiment of the invention, the system comprises:
a device according to the first or second aspect of the invention,
an interlocking element, the interlocking element adapted to be fixated to a peripheral positioning device, the interlocking element further comprising:
mounting means adapted to mount the housing on the interlocking element,
at least one sensor positioned within the interlocking element, the sensor adapted to communicate with a peripheral device, preferably the peripheral positioning device,
   wherein the at least one sensor is further adapted to sense a coaxial position of the elongate base portion of the holding element relative to the canal, the guiding mechanism is adapted to provide variable resistance between the housing and the surface of the elongate base portion of the holding element, when the holding element is translated within the canal of the housing as the associated elongate sample element obtains a sample from a subject and the system is adapted to stop the peripheral positioning device based on a signal from the at least one sensor.

In a sixth aspect, the invention relates to the use of a device according the first or second aspect of the invention or the system according to the fifth aspect of the invention for the acquisition of a biological sample from an oral or nasal cavity of a subject.

In an alternative embodiment of the invention, the use of the device or system comprises the provision of a device according to the first or second aspect of the invention or the system according to the fifth aspect of the invention, and wherein said device or system performs the method according to the fourth aspect of the invention for the obtainment of a biological sample from a subject, preferably from a nasal or oral cavity of said subject.

Any of the aspects of the present invention may each be combined with any of the other aspects. These and other aspects of the invention will be apparent from and elucidated with reference to the embodiments described hereinafter.

### BRIEF DESCRIPTION OF THE FIGURES

The device, method and system according to the invention will now be described in more detail with regard to the accompanying figures. The figures show one way of implementing the present invention and is not to be construed as being limiting to other possible embodiments falling within the scope of the attached claim set.

FIG. 1 is a cross sectional side view of the assembled device according to an embodiment of the invention.

FIG. 2A is another cross sectional side view of the assembled device according to an embodiment of the invention.

FIG. 2B is yet another cross sectional view of the assembled device according an embodiment of the invention.

FIG. 3A is an exploded trimetric view of the device according to an embodiment of the invention.

FIG. 3B is a trimetric view of the device according to FIG. 3A when assembled.

FIG. 4A is a side view of the holding element according to an embodiment of the invention.

FIG. 4B is a trimetric view of the sample element holding portion of the holding element according to an embodiment of the invention.

FIG. 5 is a cross sectional side view of the housing according to an embodiment of the invention.

FIG. 6A is a cross sectional side view of a portion of the holding element and the housing, respectively, according to an embodiment of the invention.

FIG. 6B is another cross sectional side view of a portion of the holding element and the housing, respectively, according to an embodiment of the invention.

FIG. 6C is a cross sectional frontal view of a portion of the holding element and the housing, respectively, according to an embodiment of the invention.

FIG. 7A is a cross sectional side view of a portion of the holding element and the housing, respectively, according to an alternative embodiment of the invention.

FIG. 7B is another cross sectional side view of a portion of the holding element and the housing, respectively, according to an alternative embodiment of the invention.

FIG. 8A is a cross sectional side view of a portion of the holding element and the housing, respectively, according to another alternative embodiment of the invention.

FIG. 8B is another cross sectional side view of a portion of the holding element and the housing, respectively, according to another alternative embodiment of the invention.

FIG. 9A is a cross sectional side view of a portion of the holding element and the housing, respectively, according to yet another alternative embodiment of the invention.

FIG. 9B is another cross sectional side view of a portion of the holding element and the housing, respectively, according to yet another alternative embodiment of the invention.

FIG. 10 is cross sectional side view of the holding element, according to an embodiment of the invention, when translating within the canal of the housing.

FIG. 11 is a flow-chart of a method according to an embodiment of the invention.

FIG. 12 is cross sectional side view of the holding element HE, according to another preferred embodiment of the invention.

### DETAILED DESCRIPTION OF AN EMBODIMENT

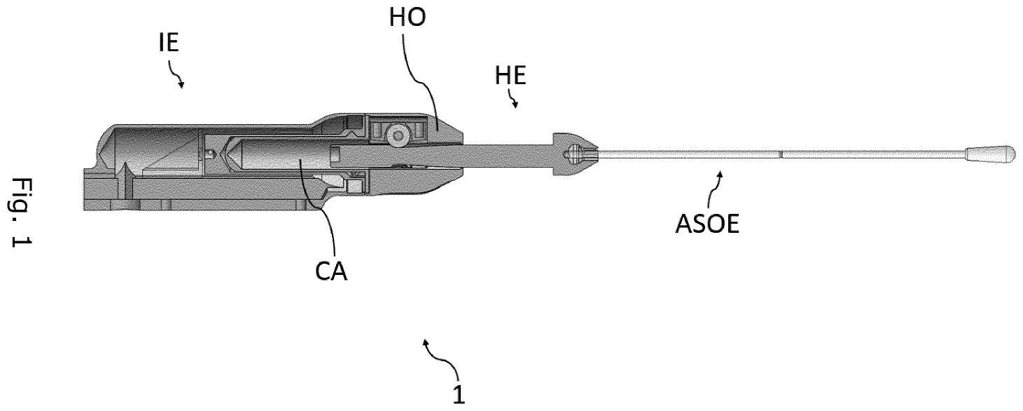

FIG. 1 is a cross sectional side view of the assembled device 1, according to an embodiment of the invention. FIG. 1 shows an assembled device 1 wherein the associated sample obtaining device ASOE is fixated to the holding element HE, the holding element HE is inserted into the canal CA of the housing HO and the housing HO is mounted partly inside the interlocking element IE.

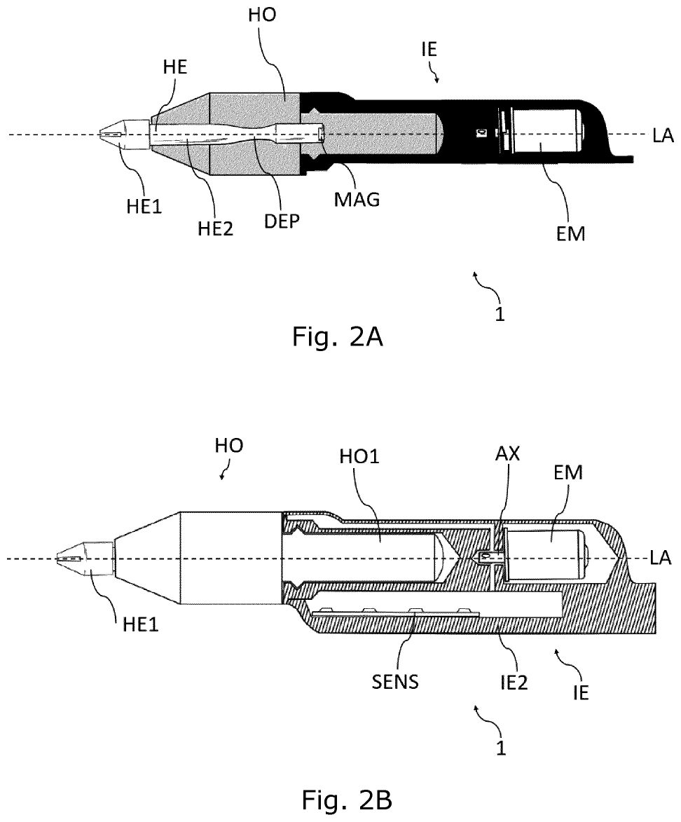

FIG. 2A is another cross sectional/semi-transparent side view of the assembled device, according to an embodiment of the invention. FIG. 2A shows an assembled device 1, wherein the elongate base portion HE2 of the holding element HE is inserted into the housing HO. Within the housing HO, the depression DEP on the surface of the elongate base portion HE2 can be seen. The sample element holding portion HE1 protrudes from the housing HO, thus enabled to receive an associated sample obtaining element (not shown). Opposite the sample element holding portion HE1, a magnetic element MAG is embedded within the elongate base portion HE2. Further, the housing HO is mounted partly within the interlocking element IE. At an end of the interlocking element IE opposite the housing HO, an electric motor EM is positioned with an axle or shaft adapted to rotate the housing HO around the longitudinal axis LA, relative to the interlocking element IE.

FIG. 2B is yet another cross sectional/semi-transparent view of the assembled device, according an embodiment of the invention. FIG. 2B shows the sample element holding portion HE1 of the holding element protruding at an end of the housing HO. The housing HO further has an elongate portion HO1 extending into the interlocking element IE. Within the interlocking element, an array of sensors SENS is positioned, adapted to sense a position of the holding element. At an end of the interlocking element IE opposite the housing HO, an electric motor EM is positioned, adapted to rotate an inner portion of the interlocking element IE wherein the housing HO is positioned, thus rotating housing HO around the longitudinal axis LA, relative to an outer periphery of the interlocking element IE.

In an alternative embodiment, the electric motor EM is positioned with an axle or shaft AX adapted to rotate the housing HO around the longitudinal axis LA, relative to the interlocking element IE.

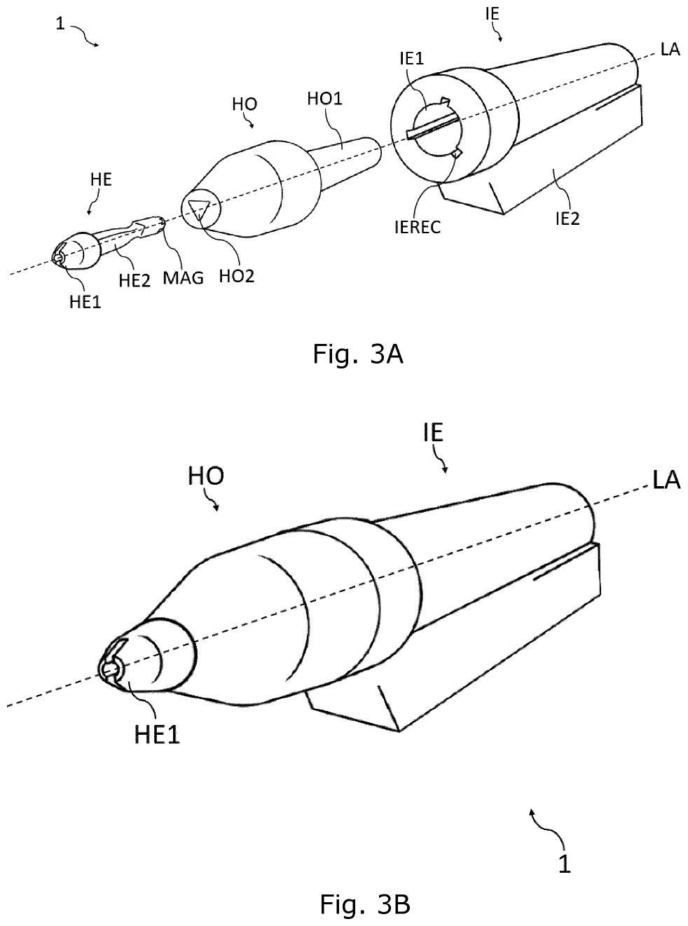

FIG. 3A is an exploded view of the device 1, according to an embodiment of the invention. FIG. 3A shows the holding element HE, the housing HE and the interlocking element IE arranged coaxially on a longitudinal axis LA, ready to assemble by translating the holding element HE and the interlocking element IE inwards, towards the housing HO, on the longitudinal axis LA (shown in FIG. 3B). At one end of the holding element HE, the sample element holding portion HE1 is arranged and at an opposite end, a magnetic element MAG is embedded within the elongate base portion HE2. At one end of the housing HO, the housing has an opening HO2, adapted to receive the elongate base portion HE2. In this particular embodiment, the opening HO2 of the housing HO has a triangular cross section, corresponding to a substantially triangular outer surface of the elongate base portion HE2. Further, a cross sectional diameter of the sample element holding portion HE1 is larger than the opening HO2 of the housing, thus preventing the holding element HE from translating fully into the housing HO. The housing HO further has an elongate portion HO1, adapted to be inserted into and mounted partly within the interlocking element IE. The interlocking element IE has a circular opening IE1 with three recesses IEREC arranged around an inner periphery of the opening IE1. Said recesses IEREC are adapted to engage with protruding element or fins (not shown) on an outer surface of the elongate portion HE1 of the housing HO and to fixate the elongate portion HE1 of the housing HO to the interlocking element IE in an axial and/or radial direction. Said fins interacting with the recesses IEREC further transfers rotation from the electric motor to the housing HO. The interlocking element IE is adapted with an interlocking portion IE2, adapted to be interlocked with a peripheral device (not shown), such as a robotic arm or other peripheral positioning device. Further, at least a portion of the housing HO has a cross sectional diameter which is larger than the opening IE1 of the interlocking element IE, thus preventing the housing HO from translating fully into the interlocking element IE. It is further to be understood, that in some embodiments, the interlocking portion IE2 of the interlocking element IE is adapted to house the at least one sensor SENS (not shown).

FIG. 3B is the device according to FIG. 3A when assembled. FIG. 3B shows the assembled device 1 wherein the sample element holding portion HE1 protrudes from the housing HO and the housing HO is mounted to or partly within the interlocking element IE.

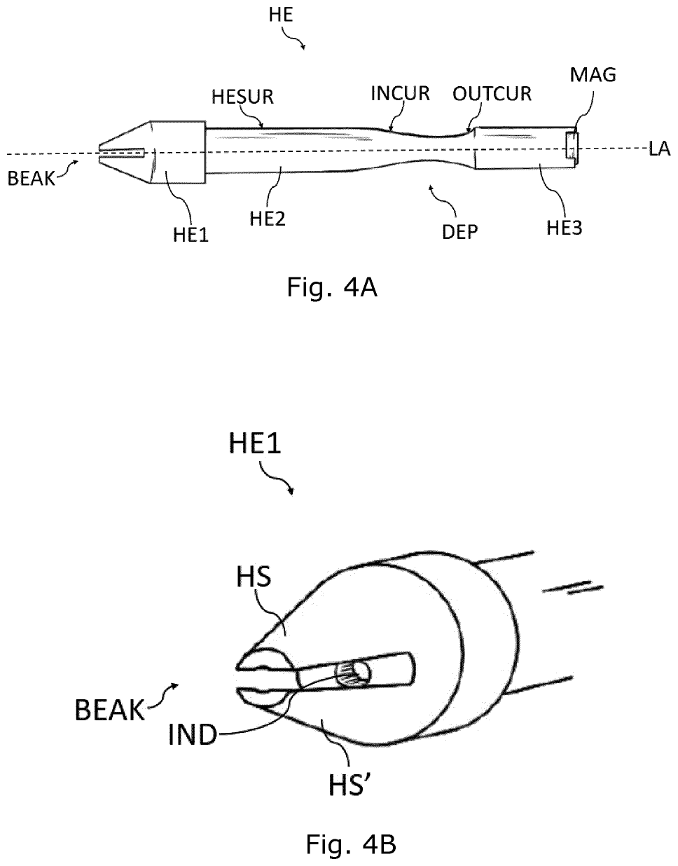

FIG. 4A is a side view of the holding element HE, according to an embodiment of the invention. FIG. 4A shows the holding element HE arranged on a longitudinal axis LA. At one end, the sample element holding portion HE1 protrudes radially outwards from the surface HESUR of the elongate base portion HE2. At an end of the sample element holding portion HE1, opposite the elongate base portion HE2, fixating beaks BEAK for fixating an associated sample obtaining element (not shown) is positioned, adapted for releasable fixation of said associated sample obtaining element. Along a portion of the elongate base portion HE2, a cross sectional diameter of the surface HESUR of the elongate base portion reduces, i.e. the surface HESUR curves radially inwards INCUR, creating a curved depression DEP. At the opposite end of the inwards curve INCUR of the depression DEP, the surface HESUR extends radially outwards, creating an outwards curvature OUTCUR, extending into a back portion of the elongate base portion HE3. Within said back portion HE3, a magnetic element MAG is embedded into the holding element HE.

FIG. 4B is a trimetric view of the sample element holding portion HE1 of the holding element. FIG. 4B shows the fixation beaks BEAK comprised of two outwardly extending half shells HS, HS′ and wherein an indentation IND is positioned, centred between said half shells HS, HS′; the indentation IND extending into a portion of the sample element holding portion HE1. It is to be understood, that the fixation beaks BEAK and the indentation IND is adapted to receive and releasably fixate an associated sample obtaining element (not shown).

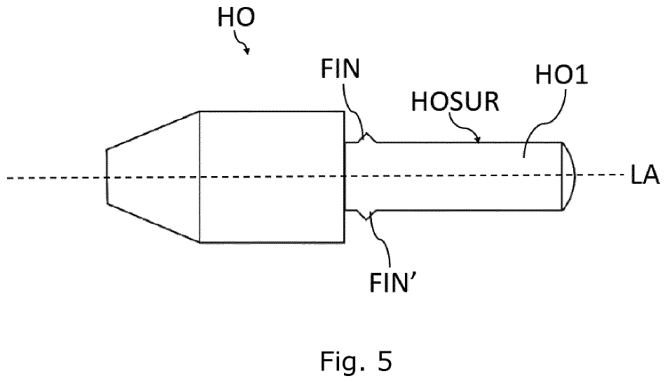

FIG. 5 is a cross sectional side view of the housing HO, according to embodiment of the invention. FIG. 5 shows the housing HO arranged on a longitudinal axis, wherein the elongate portion HO1 has an outer surface HOSUR and wherein said surface HOSUR has at least two protruding fins FIN, FIN′ adapted to engage with recesses IEREC of the opening IE1 of the interlocking element IE (not shown) so as to arrange the housing radially around the longitudinal axis LA, relative to said interlocking element IE.

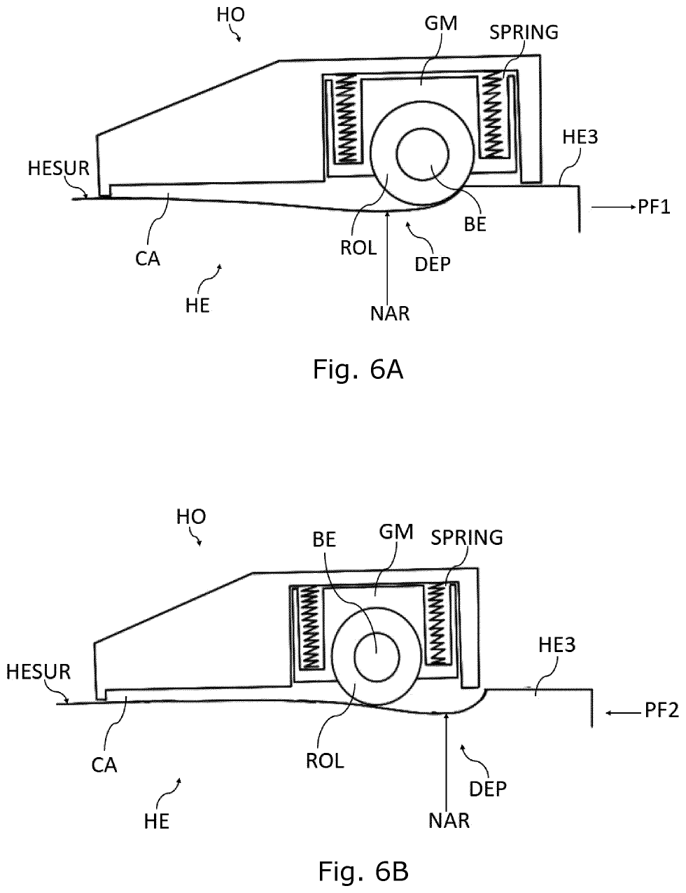

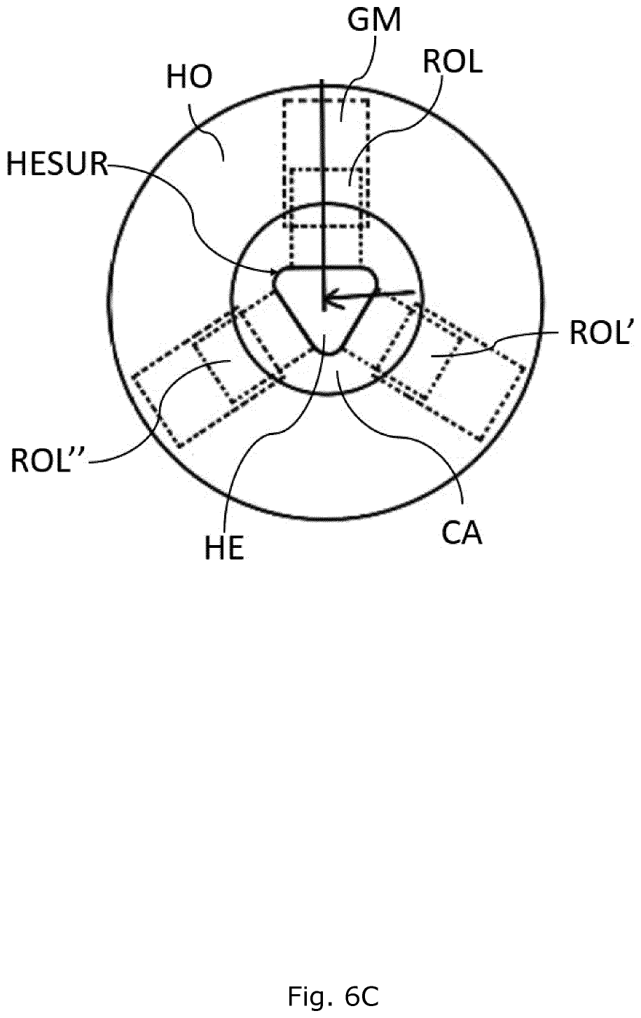

FIG. 6A is a cross sectional side view of a portion of the holding element HE and the housing HO respectively. FIG. 6A shows the interaction between the guiding mechanism GM of the housing HO and the surface HESUR and depression DEP of the holding element HE. According to a preferred embodiment of the invention, the guiding mechanism comprises spring biased rollers, wherein at least one roller ROL is situated on a bearing BE within the guiding mechanism GM, and wherein the guiding mechanism is biased with one or more springs SPRING, enabling the rollers ROL of the guiding mechanism to abut and guide the surface HESUR of the holding element HE. In this figure, the guiding mechanism GM and roller ROL is slightly offset to a narrowest point NAR of the depression DEP, and wherein a spring force of the springs SPRING performs a pull force PF1 on the holding element HE due to the roller ROL pushing on the surface HESUR of the holding element HE (OUTCUR of FIG. 4A), i.e. wherein the holding element HE is pulled further into the canal CA of the housing HO.

It is to be understood that as the holding element HE is inserted into the housing HO, the roller ROL is pushed into the guiding mechanism GM compressing the springs SPRING, due to the cross sectional diameter of the back portion HE3 of the holding element HE and wherein, as the roller ROL becomes collateral to the depression, the compression of the springs SPRING pulls the holding element HE further into the housing HO, until the roller ROL and guiding mechanism GM is collateral to narrowest point NAR of the depression DEP.

FIG. 6B is another cross sectional side view of a portion of the holding element and the housing respectively. FIG. 6B shows the interaction between the guiding mechanism GM of the housing HO and the surface HESUR and depression DEP of the holding element HE. According to a preferred embodiment of the invention, the guiding mechanism comprises spring biased rollers, wherein at least one roller ROL is situated on a bearing BE within the guiding mechanism GM, and wherein the guiding mechanism is biased with one or more springs SPRING, enabling the rollers ROL of the guiding mechanism to abut and guide the surface HESUR of the holding element HE. In this figure, the guiding mechanism GM and roller ROL is offset to a narrowest point NAR of the depression DEP, and wherein a spring force of the springs SPRING performs a push force PF2 on the holding element HE due to the roller ROL pushing on the surface HESUR of the holding element HE (INCUR of FIG. 4A), i.e. wherein the holding element HE is pushed further outwards of the canal CA of the housing HO. It is to be understood, that the resistance force required to move the holding element HE relative to the housing HO is dependent on the position of the guiding mechanism GM relative to the surface HESUR of the holding element HE.

FIG. 6C is a cross sectional/semi-transparent frontal view of a portion of the holding element and the housing respectively. FIG. 6C shows a peripheral outline of the housing HO and the holding element HE respectively, the holding element HE positioned centred within the canal CA of the housing. The surface HESUR of the holding element HE is substantially triangular with three sides, wherein the guiding mechanism GM is comprised of three corresponding sets of rollers ROL, ROL′, ROL″, each of the rollers ROL, ROL′, ROL″ abutting a side of the surface HESUR of the holding element HE.

It is to be understood, that the configuration of the three rollers ROL, ROL′, ROL″ the cross sectional triangular geometry of the surface HESUR of the holding elements provides for an radial orientation of the holding element HE with respect to the housing.

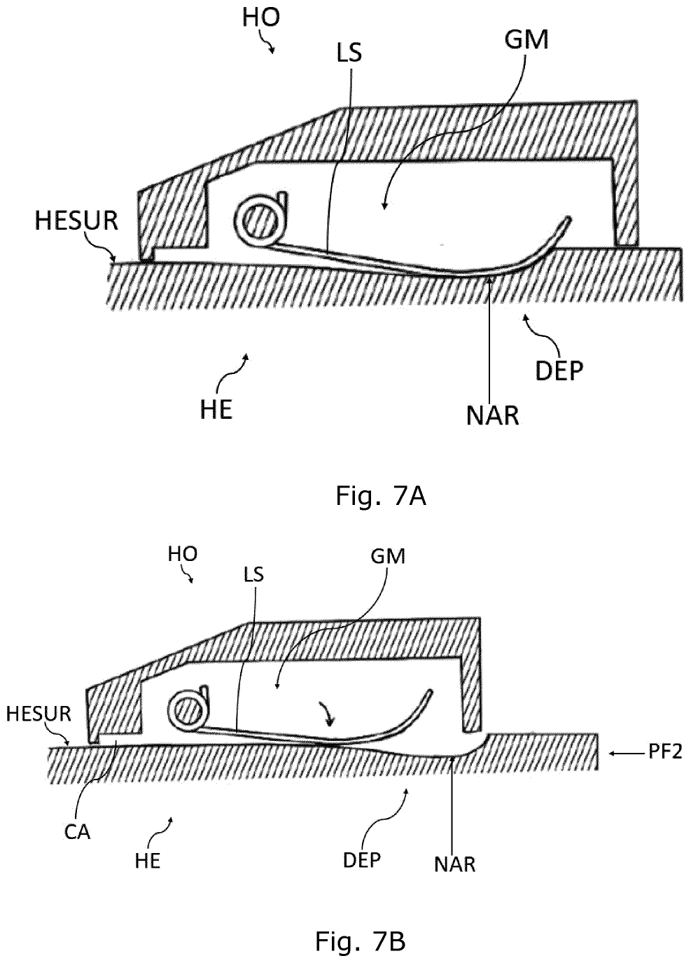

FIG. 7A is a cross sectional side view of a portion of the holding element HE and the housing HO respectively, according to an alternative embodiment of the invention. FIG. 7A shows an alternative embodiment, wherein the guiding mechanism GM of the housing HO is a leaf spring LS, abutting the surface HESUR of the holding element HE. In this figure, the leaf spring LS is collateral to the narrowest point NAR of the surface HESUR, thus the holding element HE is in a passive position, neither being forced outwards or into the housing HO.

FIG. 7B is another cross sectional side view of a portion of the holding element HE and the housing HO respectively, according to an alternative embodiment of the invention. FIG. 7B shows an alternative embodiment, wherein the guiding mechanism GM of the housing HO is a leaf spring LS, abutting the surface HESUR of the holding element HE. In this figure, the leaf spring LS is offset to the narrowest point NAR of the surface HESUR, the spring force of the leaf spring LF performing a push force PF2 on the holding element HE due to the leaf spring LS pushing on the surface HESUR of the holding element HE (INCUR of FIG. 4A), i.e. wherein the holding element HE is pushed further outwards of the canal CA of the housing HO.

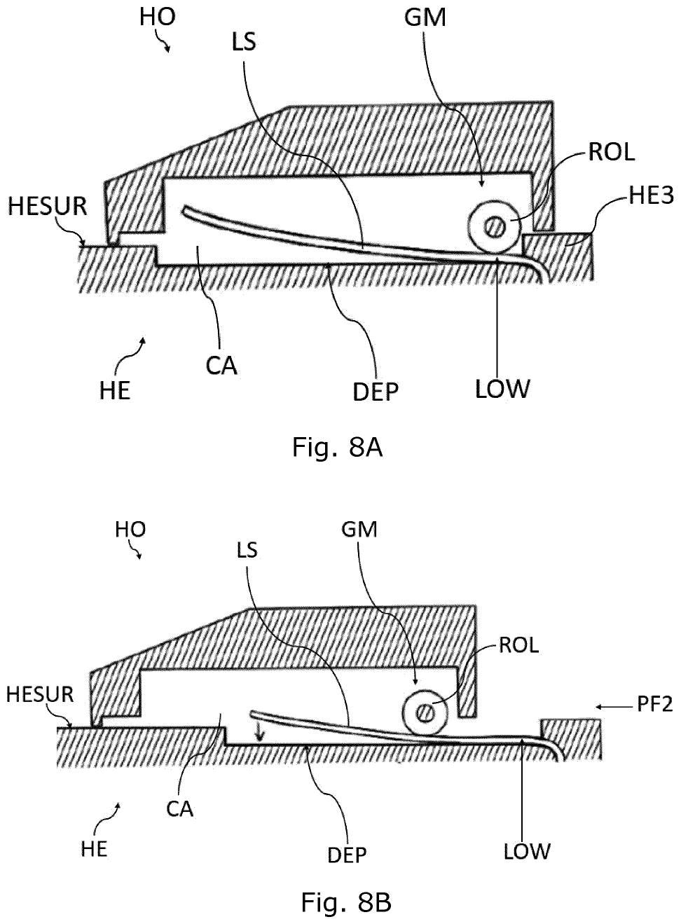

FIG. 8A is a cross sectional side view of a portion of the holding element and the housing respectively, according to another alternative embodiment of the invention. FIG. 8A shows an alternative embodiment, wherein the guiding mechanism GM of the housing HO is a roller ROL abutting a leaf spring LS, wherein the leaf spring LS is attached to a back portion HE3 of the holding element HE, the leaf spring LS extending towards the sample element holding portion (not shown) of the holding element HE and extending radially outwards from the surface HESUR of the holding element HE. Further, in this embodiment, the depression DEP of the holding element HE is non-curved, with a substantially linear surface. In this figure, the roller ROL is collateral to a lowest point LOW of the leaf spring LS, the roller ROL further resting against an edge of the back portion HE3, thus the holding element HE is in a passive position, neither being forced outwards or into the housing HO.

FIG. 8B is another cross sectional side view of a portion of the holding element and the housing respectively, according to another alternative embodiment of the invention. FIG. 8B shows an alternative embodiment, wherein the guiding mechanism GM of the housing HO is a roller ROL abutting a leaf spring LS, wherein the leaf spring LS is attached to a back portion HE3 of the holding element HE, the leaf spring LS extending towards the sample element holding portion (not shown) of the holding element HE and extending radially outwards from the surface HESUR of the holding element HE. Further, in this embodiment, the depression DEP of the holding element HE is non-curved, with a substantially linear surface. In this figure, the roller ROL is offset to a lowest point LOW of the leaf spring LS, thus a tension between the leaf spring LS and the roller ROL exerts a push force PF2, pushing the holding element HE outwards, relative to the housing HO. It is further to be understood, that in this embodiment, the position of the roller ROL is stationary relative to the housing HO, and that the leaf spring LS moves relative to said roller ROL.

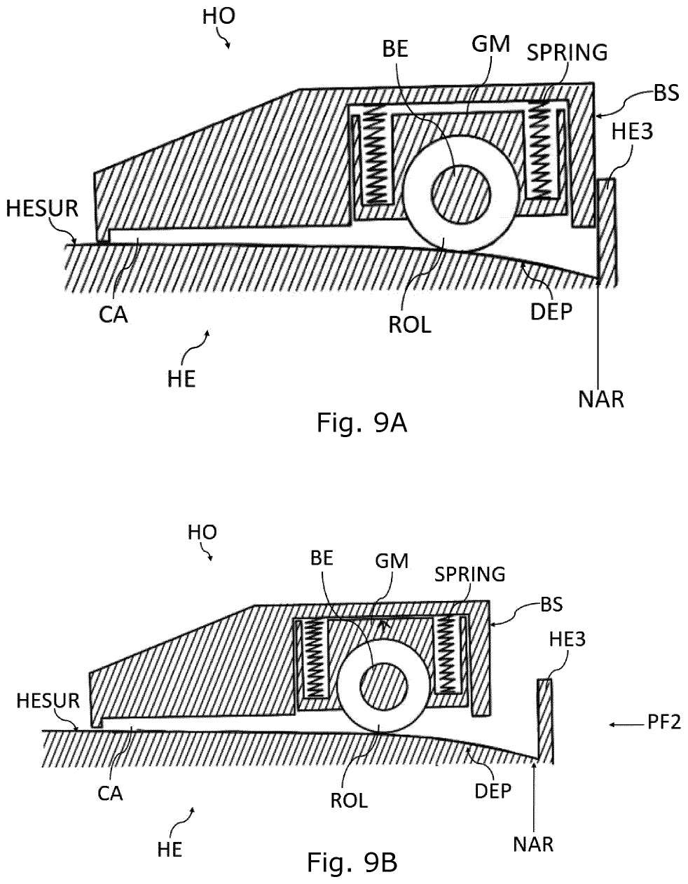

FIG. 9A is a cross sectional side view of a portion of the holding element and the housing respectively, according to yet another alternative embodiment of the invention. FIG. 9A shows the interaction between the guiding mechanism GM of the housing HO and the surface HESUR and depression DEP of the holding element HE. According to a preferred embodiment of the invention, the guiding mechanism comprises spring biased rollers, wherein at least one roller ROL is situated on a bearing BE within the guiding mechanism GM, and wherein the guiding mechanism is biased with one or more springs SPRING, enabling the rollers ROL of the guiding mechanism to abut and guide the surface HESUR of the holding element HE. In this figure, the back portion HE3 of the holding element has a cross sectional diameter which is larger, than a cross sectional diameter of at least a portion of the canal CA of the housing HO, thus providing a stop function wherein the holding element HE is prohibited from translating out of the canal CA, as the back portion HE3 abuts a back surface BS of the canal CA. From FIG. 9A it is shown, that the holding element HE is in a stationary position relative to the housing HO, due to the abutting of the back surface BS and the back portion HE3 of the holding element.

FIG. 9B is another cross sectional side view of a portion of the holding element and the housing respectively, according to yet another alternative embodiment of the invention. FIG. 9B shows the interaction between the guiding mechanism GM of the housing HO and the surface HESUR and depression DEP of the holding element HE. According to a preferred embodiment of the invention, the guiding mechanism GM comprises spring biased rollers, wherein at least one roller ROL is situated on a bearing BE within the guiding mechanism GM, and wherein the guiding mechanism is biased with one or more springs SPRING, enabling the rollers ROL of the guiding mechanism to abut and guide the surface HESUR of the holding element HE. In this figure, the back portion HE3 of the holding element has a cross sectional diameter which is larger, than a cross sectional diameter of at least a portion of the canal CA of the housing HO, thus providing a stop function wherein the holding element HE is prohibited from translating out of the canal CA, as the back portion HE3 abuts a back surface BS of the canal CA. From FIG. 9B it is shown that the compression force of the spring SPRING translates into a force between the roller ROL and the sloped surface of the depression DEP, and wherein the axial force is in the direction as shown by the arrow PF2.

Further, as shown by FIG. 9A and FIG. 9B, in some embodiments the depression is a slope, and wherein no OUTCURV as within FIG. 4A is present, and the backstop according to the geometry of the back portion HE3 instead is provided.

With respect to FIG. 6A, 6B, 7A, 7B, 8A, 8B, 9A and 9B it is to be understood that the push force PF2 equals the resistance force (RF) of the holding element HE relative to the housing HO, as the associated sample obtaining element abuts tissue of a subject and wherein the holding element HE is pushed into the housing HO.

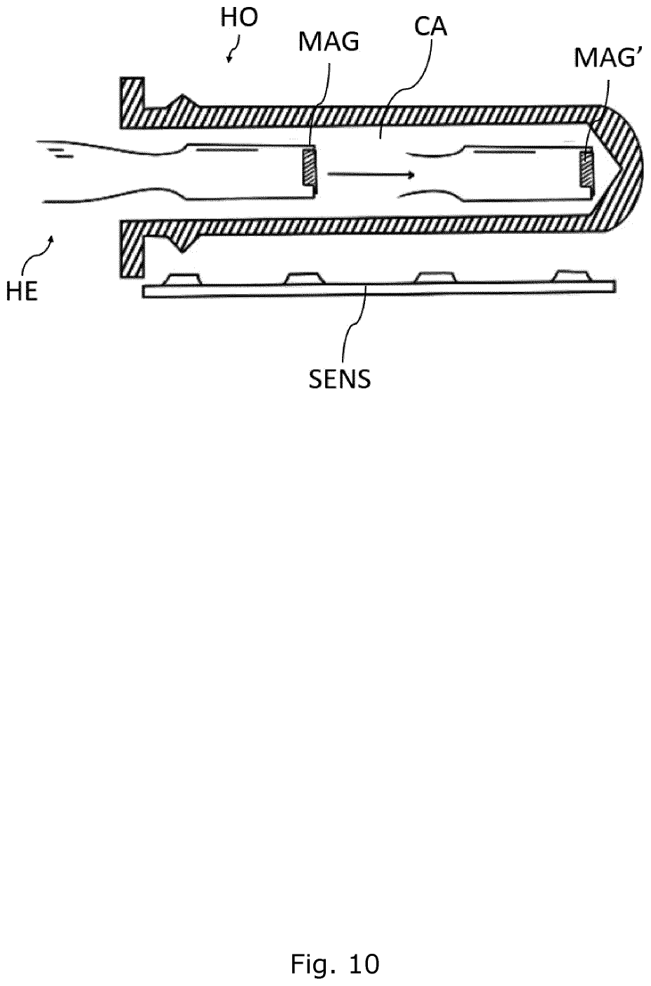

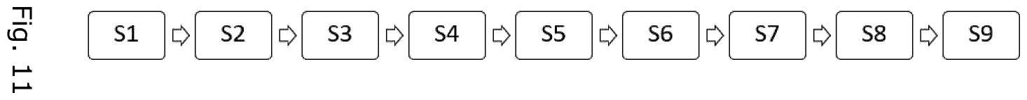

FIG. 10 is cross sectional side view of the holding element when translating within the canal of the housing. FIG. 10 shows the translation, indicated with an arrow, of the holding element HE relative to the canal CA of the housing HO, and wherein an array of sensors SENS, is arranged to measure a position of the magnet MAG, MAG′ within the holding element HE relative to the housing HO. In some embodiments, the at least one sensor SENS is arranged within the housing HO.

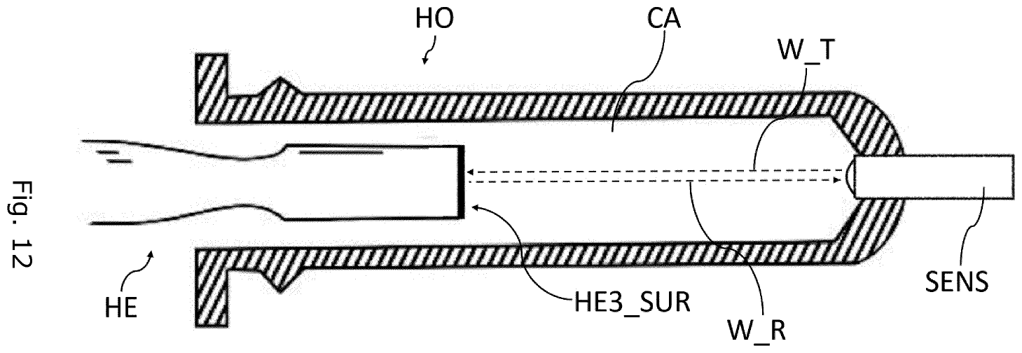

FIG. 11 is a flow-chart of a method according to the invention, the method comprising the following steps:
S1—providing an interlocking element, the interlocking element being positioned on a peripheral positioning device,
S2—providing a housing mounted on the interlocking element, the housing comprising:
   a canal with an opening, and
   a guiding mechanism,
S3—providing a holding element, the holding element comprising
   a sample element holding portion adapted to hold an associated elongate sample obtaining element, and
   an elongate base portion connected to the sample element holding portion, the elongate base portion being tapered, increasing in cross-section from an end opposite from the sample element holding portion towards the sample element holding portion and a depression at a position between the second end and the sample element holding portion,
S4—providing at least one sensor, said sensor being adapted to detect a position of the elongate base portion relative to the canal, and further adapted to communicate with a peripheral device, preferably wherein the sensor is in communication with the peripheral positioning device,
S5—fixating the associated elongate sample obtaining element to the sample element holding portion,
S6—introducing the elongate base portion into the opening of the canal until the depression of the elongate base portion is collateral with the guiding mechanism,
S7—moving the associated elongate sample obtaining element towards and into an oral or nasal cavity of a subject by moving the peripheral positioning device from a first position to at least a second position, and
S8—obtaining a sample from the oral or nasal cavity of the subject and moving the peripheral positioning device from the at least second position to a position different from the second position, and
S9—stopping the peripheral positioning device from moving based on a signal from the at least one sensor.

FIG. 12 is cross sectional side view of the holding element HE, according to another preferred embodiment of the invention. FIG. 12 shows the holding element HE relative to the canal CA of the housing HO, and wherein an optical sensor SENS, is arranged to measure the distance between said sensor SENS and a surface HE3_SUR of the holding element, said surface HE3_SUR facing towards the sensor SENS. The dotted arrows indicate waves transmitted W_T from the sensor SENS towards the surface HE3_SUR and received W_R back into the sensor, when reflected from said surface HE3_SUR. The distance may be used to calculate movement and/or speed and/or acceleration of the holding element HE relative to the housing HO, at least when providing a plurality of distance measurements relative to time passed.

In the following preferred embodiments and aspects of the invention are presented as a list of items:
Item 1. A device (1) for acquisition of a biological sample from an oral or nasal cavity of a subject, the device comprising:
   a holding element (HE) comprising:
      a sample element holding portion (HE1), adapted to releasably fixate an end of an associated elongate sample obtaining element (ASOE), such as a swab, opposite a sample obtaining end of the associated elongate sample obtaining element,
      an elongate base portion (HE2) having a longitudinal axis (LA) coaxial to a length axis of said elongate base portion, the elongate base portion being connected to the sample element holding portion at a first end of the elongate base portion, the elongate base portion further having at least a first depression (DEP) along an outer surface (HESUR),
   a housing (HO) comprising:
      a canal (CA) with an opening (HO2), wherein the opening is adapted to receive a second end (HE3) of the elongate base portion and the canal is adapted to enclose at least a portion of the elongate base portion,
      a guiding mechanism (GM) adapted to abut and guide the outer surface of the elongate base portion of the holding element within the canal,
   at least one sensor (SENS), and when the elongate base portion is positioned within the canal, the at least one sensor is adapted to sense a position of the elongate base portion of the holding element relative to the canal, and wherein the guiding mechanism is adapted to provide a resistance force RF, between the canal and the elongate base portion of the holding element when the holding element is translated within the canal of the housing, wherein the RF is coaxial to the longitudinal axis of the elongate base portion and said RF varies depending on the position of the elongate base portion relative to the canal.
Item 2. The device according to Item 1 further comprising an interlocking element (IE), the interlocking element adapted to be fixated to a peripheral positioning device, such as a robotic arm, the interlocking element further comprising mounting means (IE1, IEREC) adapted to mount the housing on the interlocking element, and wherein the at least one sensor is positioned within the interlocking element.
Item 3. The device according to Item 1 or 2, the device further comprising an electric motor (EM) adapted to rotate the housing relative to the interlocking element or the holding element relative to the housing or the associated elongate sample obtaining relative to the holding element so as to rotate the associated elongate sample obtaining element radially around a longitudinal axis (LA).
Item 4. The device according to Item 3, wherein the electric motor is adapted to rotate when a sensor, such as said at least one sensor or a second sensor, measures translation of the elongate base portion within the canal from a first position to a second position, as a result of force applied to the holding element from the associated elongate sample obtaining element, preferably wherein the electric motor is engaged when the sample obtaining end of the associated sample obtaining element abuts tissue of the subject.
Item 5. The device according to any of the preceding Items wherein the at least one sensor is an electromagnetic sensor, such as a hall sensor and the holding element further comprises a magnetic portion, the electromagnetic sensor being adapted to sense the position of the holding element within the canal of the housing.
Item 6. The device according to any of the preceding Items, wherein the elongate base portion of the holding element is tapered and wherein a cross-section of the elongate base portion in at least a section, increases from the second end towards the sample element holding portion.
Item 7. The device according to any of the preceding Items, wherein the cross-section of the elongate base portion of the holding element is noncircular, preferably the cross-section is substantially triangular and wherein the elongate base portion has three sides.
Item 8. The device according to any of the preceding Items, wherein the guiding mechanism comprises at least a first and second set of rollers (ROL, ROL′) adapted to roll along the surface of the elongate base portion of the holding element, preferably wherein three sets of rollers (ROL, ROL′, ROL″) abuts the three sides of the elongate base portion according to claim 7.
Item 9. The device according to Item 8, wherein the at least first set of rollers are spring biased (SPRING) towards the surface of the elongate base portion of the holding element, with a spring force SF substantially lateral to the longitudinal axis of the elongate base portion, wherein the SF translates to the RF, an application force AF is coaxial to the longitudinal axis of the elongate base portion in a direction opposite to the RF, and to insert the elongate base portion into the canal of the housing, the AF exceeds the RF.
Item 10. The device according to Item 9, wherein the frictional force FF is electronically adjustable by providing braking means for at least one of the at least first and second set of rollers.
Item 11. The device according to any of the preceding Items, further comprising an output device connected to the at least one sensor, the output device adapted to send a signal to a peripheral device based on a measurement from said at least one sensor.
Item 12. The device according to any of the preceding Items, wherein the holding element and the elongate sample obtaining element are one unitary element.
Item 13. A method of acquiring a biological sample from an oral or nasal cavity of a subject, the method comprising:
   providing an interlocking element (IE), the interlocking element being positioned on a peripheral positioning device,
   providing a housing (HO) mounted on the interlocking element, the housing comprising:
      a canal (CA) with an opening (HO2), and
      a guiding mechanism (GM),
   providing a holding element (HE) comprising:
      a sample element holding portion (HE1) adapted to hold an associated elongate sample obtaining element (ASOE), and
      an elongate base portion (HE2) connected to the sample element holding portion, the elongate base portion being tapered, increasing in cross-section from a second end (HE3) opposite from the sample element holding portion towards the sample element holding portion and a depression (DEP) at a position between the second end and the sample element holding portion,
   providing at least one sensor (SENS), said sensor being adapted to detect a position of the elongate base portion relative to the canal, and further adapted to communicate with a peripheral device, preferably wherein the sensor is in communication with the peripheral positioning device,
   fixating the associated elongate sample obtaining element to the sample element holding portion,
   introducing the elongate base portion into the opening of the canal until the depression of the elongate base portion is collateral with the guiding mechanism,
   moving the associated elongate sample obtaining element towards and into an oral or nasal cavity of a subject by moving the peripheral positioning device from a first position to at least a second position, and
   obtaining a sample from the oral or nasal cavity of the subject and moving the peripheral positioning device from the at least second position to a position different from the second position, and
   stopping the peripheral positioning device from moving based on a signal from the at least one sensor.
Item 14. The method according to Item 13, further comprising:
   providing an electric motor (EM) adapted to rotate the housing relative to the interlocking element or the holding element relative to the housing or the associated elongate sample obtaining element relative to the holding element, and
   activating the electric motor when the peripheral positioning device is at least in the second position.
Item 15. A system for acquisition of a biological sample from an oral or nasal cavity of a subject, the system comprising:
   a holding element (HE) comprising:
      a sample element holding portion (HE1), adapted to releasably fixate an end of an associated elongate sample obtaining element (ASOE) opposite a sample obtaining end of the associated elongate sample obtaining element,
      an elongate base portion (HE2) connected to the sample element holding portion at a first end of the elongate base portion, the elongate base portion further having at least a first depression (DEP) along an outer surface (HESUR),
   a housing (HO) comprising:
      a canal (CA) with an opening (HO2), wherein the opening is adapted to receive a second end (HE3) of the elongate base portion and the canal is adapted to enclose at least a portion of the elongate base portion,
      a guiding mechanism (GM) adapted to abut and guide the outer surface of the elongate base portion of the holding element within the canal,
   an interlocking element (IE), the interlocking element adapted to be fixated to a peripheral positioning device, the interlocking element further comprising:
      mounting means (IE1, IEREC) adapted to mount the housing to the interlocking element,
      at least one sensor (SENS) positioned within the interlocking element, the sensor adapted to communicate with a peripheral device, preferably the peripheral positioning device,
   wherein the at least one sensor is further adapted to sense a coaxial position of the elongate base portion of the holding element relative to the canal, the guiding mechanism is adapted to provide variable resistance between the housing and the surface of the elongate base portion of the holding element when the holding element is translated within the canal of the housing, as the associated elongate sample element obtains a sample from a subject and the system is adapted to stop the peripheral positioning device based on a signal from the at least one sensor.
Item 2.1. A device (1) for acquisition of a biological sample from an oral or nasal cavity of a subject, the device comprising:
   a holding element (HE) comprising:
      a sample element holding portion (HE1), adapted to releasably fixate an end of an associated elongate sample obtaining element (ASOE), such as a swab, opposite a sample obtaining end of the associated elongate sample obtaining element,
      an elongate base portion (HE2) having a longitudinal axis (LA) coaxial to a length axis of said elongate base portion, the elongate base portion being connected to the sample element holding portion at a first end of the elongate base portion, the elongate base portion further having at least a first depression (DEP) along an outer surface (HESUR),
   a housing (HO) comprising:
      a canal (CA) with an opening (HO2), wherein the opening is adapted to receive a second end (HE3) of the elongate base portion and the canal is adapted to enclose at least a portion of the elongate base portion,
      a guiding mechanism (GM) adapted to abut and guide the outer surface of the elongate base portion of the holding element within the canal,
   at least one sensor (SENS), and when the elongate base portion is positioned within the canal,
   the at least one sensor is adapted to sense a position of the elongate base portion of the holding element relative to the canal, and wherein the guiding mechanism is adapted to provide a resistance force RF, between the canal and the elongate base portion of the holding element when the holding element is translated within the canal of the housing, wherein the RF is coaxial to the longitudinal axis of the elongate base portion and said RF varies depending on the position of the elongate base portion relative to the canal.
Item 2.2. The device according to Item 2.1 further comprising an interlocking element (IE), the interlocking element adapted to be fixated to a peripheral positioning device, such as a robotic arm, the interlocking element further comprising mounting means (IE1, IEREC) adapted to mount the housing on the interlocking element, and wherein the at least one sensor is positioned within the interlocking element.
Item 2.3. The device according to Item 2.1 or 2.2, the device further comprising an electric motor (EM) adapted to rotate the housing relative to the interlocking element or the holding element relative to the housing or the associated elongate sample obtaining relative to the holding element so as to rotate the associated elongate sample obtaining element radially around a longitudinal axis (LA).
Item 2.4. The device according to Item 2.3, wherein the electric motor is adapted to rotate when a sensor, such as said at least one sensor or a second sensor, measures translation of the elongate base portion within the canal from a first position to a second position, as a result of force applied to the holding element from the associated elongate sample obtaining element, preferably wherein the electric motor is engaged when the sample obtaining end of the associated sample obtaining element abuts tissue of the subject.
Item 2.5. The device according to any of Items 2.1 to 2.4 wherein the at least one sensor is an electromagnetic sensor, such as a hall sensor and the holding element further comprises a magnetic portion, the electromagnetic sensor being adapted to sense the position of the holding element within the canal of the housing.
Item 2.6. The device according to any of Items 2.1 to 2.5, wherein the elongate base portion of the holding element is tapered and wherein a cross-section of the elongate base portion in at least a section, increases from the second end towards the sample element holding portion.
Item 2.7. The device according to any of Items 2.1 to 2.6, wherein the cross-section of the elongate base portion of the holding element is non-circular, preferably the cross-section is substantially triangular and wherein the elongate base portion has three sides.
Item 2.8. The device according to any of Items 2.1 to 2.7, wherein the guiding mechanism comprises at least a first and second set of rollers (ROL, ROL′) adapted to roll along the surface of the elongate base portion of the holding element, preferably wherein three sets of rollers (ROL, ROL′, ROL″) abuts the three sides of the elongate base portion according to claim 7.
Item 2.9. The device according to Item 2.8, wherein the at least first set of rollers are spring biased (SPRING) towards the surface of the elongate base portion of the holding element, with a spring force SF substantially lateral to the longitudinal axis of the elongate base portion, wherein the SF translates to the RF, an application force AF is coaxial to the longitudinal axis of the elongate base portion in a direction opposite to the RF, and to insert the elongate base portion into the canal of the housing, the AF exceeds the RF.
Item 2.10. The device according to Item 2.9, wherein the frictional force FF is electronically adjustable by providing braking means for at least one of the at least first and second set of rollers.
Item 2.11. The device according to any of Items 2.1 to 2.10, further comprising an output device connected to the at least one sensor, the output device adapted to send a signal to a peripheral device based on a measurement from said at least one sensor.
Item 2.12. The device according to any of Items 2.1 to 2.11, wherein the holding element and the elongate sample obtaining element are one unitary element.
Item 2.13. The device according to any of Items 2.1 to 2.12 further comprising a processor in data connection with at least the at least one sensor.
Item 2.14. A method of acquiring a biological sample from an oral or nasal cavity of a subject, the method comprising:
   providing an interlocking element (IE), the interlocking element being positioned on a peripheral positioning device,
   providing a housing (HO) mounted on the interlocking element, the housing comprising:
      a canal (CA) with an opening (HO2), and
      a guiding mechanism (GM),
   providing a holding element (HE) comprising:
      a sample element holding portion (HE1) adapted to hold an associated elongate sample obtaining element (ASOE), and
      an elongate base portion (HE2) connected to the sample element holding portion, the elongate base portion being tapered, increasing in cross-section from a second end (HE3) opposite from the sample element holding portion towards the sample element holding portion and a depression (DEP) at a position between the second end and the sample element holding portion,
   providing at least one sensor (SENS), said sensor being adapted to detect a position of the elongate base portion relative to the canal, and further adapted to communicate with a peripheral device, preferably wherein the sensor is in communication with the peripheral positioning device,
   fixating the associated elongate sample obtaining element to the sample element holding portion,
   introducing the elongate base portion into the opening of the canal until the depression of the elongate base portion is collateral with the guiding mechanism,
   moving the associated elongate sample obtaining element towards and into an oral or nasal cavity of a subject by moving the peripheral positioning device from a first position to at least a second position, and
   obtaining a sample from the oral or nasal cavity of the subject and moving the peripheral positioning device from the at least second position to a position different from the second position, and
   stopping the peripheral positioning device from moving based on a signal from the at least one sensor.
Item 2.15. The method according to Item 2.14 comprising the further step of:
   providing a processor in data connection with one or more of the at least one sensor and the peripheral positioning device.
Item 2.16. The method according to Item 2.13, further comprising:
   providing an electric motor (EM) adapted to rotate the housing relative to the interlocking element or the holding element relative to the housing or the associated elongate sample obtaining element relative to the holding element, and
   activating the electric motor when the peripheral positioning device is at least in the second position.
Item 2.17. A system for acquisition of a biological sample from an oral or nasal cavity of a subject, the system comprising:
   the device according to any of Items 2.1 to 2.13,
   a peripheral positioning device, and
   a processor in data connection with one or more of the at least one sensor and the peripheral positioning device, the processor being adapted to control the peripheral position device based on at least a signal from the at least one sensor.
Item 2.18 The system according to Item 2.17 wherein the at least one sensor is further adapted to sense a coaxial position of the elongate base portion of the holding element relative to the canal, the guiding mechanism is adapted to provide variable resistance between the housing and the surface of the elongate base portion of the holding element when the holding element is translated within the canal of the housing, as the associated elongate sample element obtains a sample from a subject and the processor is adapted to stop the peripheral positioning device based on a signal from the at least one sensor.
Item 2.19. The system according to Item 2.17, wherein the peripheral positioning device is a robotic manipulator.
Item 2.20. Use of the device according to any of Items 2.1 to 2.13, for the acquisition of a biological sample.
Item 2.21. The use of the device according to Item 2.20, wherein the biological sample is acquisitioned from a nasal and/or oral cavity of a subject.
Item 2.22. The device according to any of Items 2.1 to 2.4, wherein the at least one sensor is an optical sensor, such as an optical sensor configured to detect the reflection of a modulated light beam and calculating distance as a function of Time-of-Flight principle, the optical sensor being adapted to sense the position of the holding element within the canal of the housing.
Item 2.23. The device according to Item 2.6, the holding element further comprising a surface normal to the modulated light beam.

In short, the invention relates to a device 1 adapted to provide safe and consistent, sample obtaining from the nasal or oral cavity of a subject. The device 1 comprises a holding element HE with an elongate base portion HE2 adapted to be mounted in a housing HO, the device 1 further comprising at least one sensor SENS, the sensor being adapted to measure a position of the elongate base portion when inserted into a canal CA of the housing. The housing further comprises a guiding mechanism GM positioned within the canal, adapted to guide and abut an outer surface HESUR of the elongate base portion HE2 of the holding element HE. It is to be understood, that consistent is to be understood as wherein the device 1 exerts a consistent pressure to the tissue of the subject according to settings of the guiding mechanism GM, such as wherein the guiding mechanism GM is adapted with a specific friction or resistance force, and wherein, when an applied pressure translating from an associated sample obtaining element ASOE to the holding element HE is exceeded, the elongate base portion HE2 of the holding element HE translates into the canal CA of the housing HO.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1. A device (1) for acquisition of a biological sample from an oral or nasal cavity of a subject, the device comprising:
a holding element (HE) comprising:
a sample element holding portion (HE1), adapted to releasably fixate an end of an associated elongate sample obtaining element (ASOE), such as a swab, opposite a sample obtaining end of the associated elongate sample obtaining element,
an elongate base portion (HE2) having a longitudinal axis (LA) coaxial to a length axis of said elongate base portion, the elongate base portion being connected to the sample element holding portion at a first end of the elongate base portion, the elongate base portion further having at least a first depression (DEP) along an outer surface (HESUR),
a housing (HO) comprising:
a canal (CA) with an opening (HO2), wherein the opening is adapted to receive a second end (HE3) of the elongate base portion and the canal is adapted to enclose at least a portion of the elongate base portion,
a guiding mechanism (GM) adapted to abut and guide the outer surface of the elongate base portion of the holding element within the canal,
at least one sensor (SENS), and when the elongate base portion is positioned within the canal, the at least one sensor is adapted to sense a position of the elongate base portion of the holding element relative to the canal, and wherein the guiding mechanism is adapted to provide a resistance force RF, between the canal and the elongate base portion of the holding element when the holding element is translated within the canal of the housing, wherein the RF is coaxial to the longitudinal axis of the elongate base portion and said RF varies depending on the position of the elongate base portion relative to the canal.

2. The device according to claim 1 further comprising an interlocking element (IE), the interlocking element adapted to be fixated to a peripheral positioning device, the interlocking element further comprising mounting means (IE1, IEREC) adapted to mount the housing on the interlocking element, and wherein the at least one sensor is positioned within the interlocking element.

3. The device according to claim 2, the device further comprising an electric motor (EM) adapted to rotate the housing relative to the interlocking element or the holding element relative to the housing or the associated elongate sample obtaining relative to the holding element so as to rotate the associated elongate sample obtaining element radially around a longitudinal axis (LA).

4. The device according to claim 3, wherein the electric motor is adapted to rotate when a sensor, such as said at least one sensor or a second sensor, measures translation of the elongate base portion within the canal from a first position to a second position, as a result of force applied to the holding element from the associated elongate sample obtaining element, optionally wherein the electric motor is engaged when the sample obtaining end of the associated sample obtaining element abuts tissue of the subject.

5. The device according to claim 1, wherein the at least one sensor is an electromagnetic sensor, and the holding element further comprises a magnetic portion, the electromagnetic sensor being adapted to sense the position of the holding element within the canal of the housing.

6. The device according to claim 1, wherein the at least one sensor is an optical sensor, such as an optical sensor configured to detect reflection of a modulated light beam and calculating distance as a function of Time-of-Flight principle, the optical sensor being adapted to sense the position of the holding element within the canal of the housing.

7. The device according to claim 6, the holding element further comprising a surface normal to the modulated light beam.

8. The device according to claim 1, wherein the elongate base portion of the holding element is tapered and wherein a cross-section of the elongate base portion in at least a section, increases from the second end towards the sample element holding portion.

9. The device according to claim 1, wherein a cross-section of the elongate base portion of the holding element is non-circular.

10. The device according to claim 1, wherein the guiding mechanism comprises at least a first and second set of rollers (ROL, ROL′) adapted to roll along the surface of the elongate base portion of the holding element.

11. The device according to claim 10, wherein the at least first set of rollers are spring biased (SPRING) towards the surface of the elongate base portion of the holding element, with a spring force SF substantially lateral to the longitudinal axis of the elongate base portion, wherein the SF translates to the RF, an application force AF is coaxial to the longitudinal axis of the elongate base portion in a direction opposite to the RF, and to insert the elongate base portion into the canal of the housing, the AF exceeds the RF.

12. The device according to claim 11, wherein a frictional force FF is electronically adjustable by providing braking means for at least one of the at least first and second set of rollers.

13. The device according to claim 1, wherein any one or more of (a) the device further comprises an output device connected to the at least one sensor, the output device adapted to send a signal to a peripheral device based on a measurement from said at least one sensor, (b) the holding element and the elongate sample obtaining element are one unitary element, or (c) the device further comprises a processor in data connection with at least the at least one sensor.

14. (canceled)

15. (canceled)

16. A method of acquiring a biological sample from an oral or nasal cavity of a subject, the method comprising:
providing an interlocking element (IE), the interlocking element being positioned on a peripheral positioning device,
providing a housing (HO) mounted on the interlocking element, the housing comprising:
a canal (CA) with an opening (HO2), and
a guiding mechanism (GM),
providing a holding element (HE) comprising:
a sample element holding portion (HE1) adapted to hold an associated elongate sample obtaining element (ASOE), and
an elongate base portion (HE2) connected to the sample element holding portion, the elongate base portion being tapered, increasing in cross-section from a second end (HE3) opposite from the sample element holding portion towards the sample element holding portion and a depression (DEP) at a position between the second end and the sample element holding portion,
providing at least one sensor (SENS), said sensor being adapted to detect a position of the elongate base portion relative to the canal, and further adapted to communicate with a peripheral device, preferably wherein the sensor is in communication with the peripheral positioning device,
fixating the associated elongate sample obtaining element to the sample element holding portion,
introducing the elongate base portion into the opening of the canal until the depression of the elongate base portion is collateral with the guiding mechanism,
moving the associated elongate sample obtaining element towards and into an oral or nasal cavity of a subject by moving the peripheral positioning device from a first position to at least a second position, and
obtaining a sample from the oral or nasal cavity of the subject and moving the peripheral positioning device from the at least second position to a position different from the second position, and
stopping the peripheral positioning device from moving based on a signal from the at least one sensor.

17. The method according to claim 16, further comprising:
providing a processor in data connection with one or more of the at least one sensor and the peripheral positioning device.

18. The method according to claim 16, further comprising:
providing an electric motor (EM) adapted to rotate the housing relative to the interlocking element or the holding element relative to the housing or the associated elongate sample obtaining element relative to the holding element, and
activating the electric motor when the peripheral positioning device is at least in the second position.

19. A system for acquisition of a biological sample from an oral or nasal cavity of a subject, the system comprising:
the device according to claim 1,
a peripheral positioning device, and
a processor in data connection with one or more of the at least one sensor and the peripheral positioning device, the processor being adapted to control the peripheral position device based on at least a signal from the at least one sensor.

20. The system according to claim 19, wherein the at least one sensor is further adapted to sense a coaxial position of the elongate base portion of the holding element relative to the canal, the guiding mechanism is adapted to provide variable resistance between the housing and the surface of the elongate base portion of the holding element when the holding element is translated within the canal of the housing, as the associated elongate sample element obtains a sample from a subject and the processor is adapted to stop the peripheral positioning device based on a signal from the at least one sensor.

21. The system according to claim 19, wherein the peripheral positioning device is a robotic manipulator.

22. A method, comprising: acquisition of a biological sample from a subject with a device according to claim 1, the biological sample optionally being acquisitioned from a nasal and/or oral cavity of a subject.

23. (canceled)
